(19) United States
(12) Patent Application Publication
(10) Pub. No.: US 2022/0288038 A1
(43) Pub. Date: Sep. 15, 2022

(54) ABCC11 INHIBITOR

(71) Applicant: THE UNIVERSITY OF TOKYO, Tokyo (JP)

(72) Inventors: Yu TOYODA, Tokyo (JP); Tappei TAKADA, Tokyo (JP); Hiroshi SUZUKI, Tokyo (JP)

(73) Assignee: THE UNIVERSITY OF TOKYO, Tokyo (JP)

(21) Appl. No.: 17/636,151

(22) PCT Filed: Aug. 21, 2020
(86) PCT No.: PCT/JP2020/031672

§ 371 Date: Feb. 17, 2022

Related U.S. Application Data
(60) Provisional application No. 62/890,053, filed on Aug. 21, 2019.

(51) Int. Cl. A61K 31/426; A61K 31/4196; A61K 8/49; A61Q 15/00; A61P 17/00

(52) U.S. Cl. CPC A61K 31/426; A61K 31/4196; A61K 8/496; A61Q 15/00; A61P 17/00

(57) ABSTRACT

This invention provides an inhibitor of multidrug resistance-associated proteins with 12 transmembrane domains containing, as an active ingredient, a compound for treatment of hyperuricemia or a salt thereof. In an embodiment, the compound is represented by Formula I

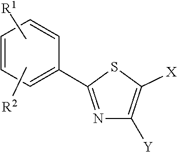

wherein R¹ represents a halogen atom, a nitro group (—NO₂), a cyano group (—CN), a formyl group (—CHO), or a trifluoromethyl group (—CF₃), R² represents a hydrogen atom or an alkoxy group having C₁₋₁₀ linear or branched alkyl group, X represents a carboxyl group (—CO₂H), a carbamoyl group (—CONH₂), or an alkoxycarbonyl group having C₁₋₅ linear or branched alkoxy group, and Y represents a hydrogen atom or a C₁₋₄ linear or branched alkyl group. In an embodiment, the multidrug resistance-associated protein with 12 transmembrane domains is ABCC11. In particular, the inhibitor of the present invention can be used in a pharmaceutical or cosmetic composition for the prevention or treatment of axillary osmidrosis.

Humans produce body odor like other animals; nonetheless, in our daily life, strong or specific body odors are sometimes perceived as an undesired constitution. Axillary osmidrosis (AO) is a condition characterized by unpleasant body odors and excessing sweating from the armpits (Wu et al., 1994). As is often the case with Asian countries where persons with strong body odor comprise a minor population, AO tends to be more strongly disliked and recognized as a disease (Toyoda et al., 2016). However, excepting surgical treatments, there was no causal therapy for AO.

Accumulating evidence suggests that ATP-binding cassette transporter C11 (ABCC11) is an AO risk factor (Nakano et al., 2009; Toyoda et al., 2009; Inoue et al., 2010; Martin et al., 2010). Although physiological substrates of ABCC11 remain unclarified, given that ABCC11-deficient subjects have little risk of AO (Ishikawa et al., 2013), together with the presence of functional ABCC11 in the axillary apocrine glands that secrete a variety of odor precursors (Toyoda et al., 2017), inhibition of ABCC11 function can contribute to the prevention and treatment of AO. However, little information has been available for ABCC11 inhibitors; no agent is approved yet for this unpleasant constitution. Here, we serendipitously identified febuxostat a well-used agent for hyperuricemia—as an ABCC11 inhibitor (FIG. 1).

Using an in vitro transport system with ABCC11-expresssing plasma. membrane vesicles (FIG. 1A), we investigated the effect of the test compound on ABCC11 function. To confirm the experimental system, we first examined the time-dependent increase in ATP-dependent dehydroepiandrosterone sulfate (DHEAS)—an ABCC11 substrate (Chen et al., 2005)—transport into the ABCC11-expressing plasma membrane vesicles (FIG. 1B). Since febuxostat inhibited ABCC11 in our preliminary experiments in vitro, we determined the IC₅₀ value of febuxostat against the ABCC11-mediated DHEAS transport activity in the presence of several concentrations of febuxostat (FIG. 1C).

We herein demonstrated that febuxostat—an approved drug on the market—is a novel ABCC11 inhibitor. Given that axillary apocrine glands open into the hair follicles, dermal administration of febuxostat via the armpits like a medical cream inhibits ABCC11 in humans, if such treatment could ensure appropriate levels of febuxostat in the ABCC11-expressing apocrine glands. Since febuxostat is a relatively safe drug, this notion will be worth considering as a future issue. Besides, investigation of ABCC11 inhibitory activities of febuxostat analogues would be encouraged. We believe that our results may shed light on a possibility of overcoming of AO in terms of drug repurposing.

### Materials and Methods

Critical materials and resources used in this study were summarized in Supplementary Table S1. Plasma membrane vesicle preparation from adenovirus mediated-transiently ABCC11-expressing 293A cells, immunoblotting, and in vitro [1,2,6,7-³H(N)]-DHEAS transport assay with ABCC11-expressing plasma membrane vesicles were conducted as described previously (Toyoda et al., 2017). Detailed description of Materials and Methods is provided in Supplementary Information.

### Conflict of Interest

The authors declare that they have no conflict of interest.

### Author Contributions

Conceptualization, YT; methodology, YT; resources, YT; validation, YT; formal. analysis, YT; investigation, YT; writing—original draft preparation, YT; writing—review and editing, YY, TT, and ITS; supervision, I-IS; project administration, YT; funding acquisition, YT.

### Funding

The study performed by the authors was supported by JSPS KAKENHI Grant Numbers 19K16441 (to YT).

### Supplementary Material

The Supplementary Material for this article can be found online.

### Data Availability Statement

Data supporting the findings of this study are included in this published article and its Supplementary Information or are available from the corresponding author on reasonable request.

### REFERENCES

Chen, Z. S., Guo, Y., Belinsky, M. G., Kotova, E., and Kruh, G. D. (2005), Transport of bile acids, sulfated steroids, estradiol 17-beta-D-glucuronide, and leukotriene C4 by human multidrug resistance protein 8 (ABCC11). Mol Pharmacol 67(2), 545-557. doi: 10.1124/mol.104.007138.

Inoue, Y., Mori, T., Toyoda, Y., Sakurai, A., Ishikawa, Mitani, Y., et al. (2010). Correlation of axillary osmidrosis to a SNP in the ABCC11 gene determined by the Smart Amplification Process (SmartAmp) method. J Plast Reconstr Aesthet Sorg 63(8), 1369-1374. doi: 10.1016/j.bjps.2009.06.029.

Ishikawa, T., Toyoda, Y., Yoshiura, K., and Niikawa, N. (2013). Pharmacogenetics of human ABC transporter ABCC11: new insights into apocrine gland growth and metabolite secretion. Front Genet 3, 306. doi: 10.3389/fgene.2012.00306.

Martin, A., Saathoff, M., Kuhn, F., Max, H., Terstegen, L., and Natsch, A. (2010). A functional ABCC11 allele is essential in the biochemical formation of human axillary odor. J Invest Dermatol 130(2), 529-540. doi: 10.1038/jid.2009.254.

Miyata, H., Takada., T., Toyoda, Y., Matsuo, H., Ichida, K., and Suzuki, H. (2016). Identification of Febuxostat as a New Strong ABCG2 Inhibitor: Potential Applications and Risks in Clinical Situations. Front Pharmacol 7, 518. doi: 10.3389/fphar.2016.00518.

Nakano, M., Miwa, N., Hirano, A., Yoshiura, K., and Niikawa, N. (2009). A strong association of axillary osmidrosis with the wet earwax type determined by genotyping of the ABCC11 gene. BMC Genet 10, 42. doi: 10.1186/1471-2156-10-42.

Toyoda, Y., Gomi, T., Nakagawa, H., Nagakura, M., and Ishikawa, T. (2016). Diagnosis of Human Axillary Osmidrosis by Genotyping of the Human ABCC11 Gene: Clinical Practice and Basic Scientific Evidence. Biomed Res Int 2016, 7670483. doi: 10.1155/2016/7670483.

Toyoda, Y., Sakurai, A., Milani, Y., Nakashima, M., Yoshiura, K., Nakagawa, H., et al. (2009). Earwax, osmidrosis, and breast cancer: why does one SNP (538G>A) in the human ABC transporter ABCC11 gene determine earwax type? FASEB J 23(6), 2001-2013. doi: 10.1096/fj.09-129098.

Toyoda, Y., Takada, T., Gomi, T., Nakagawa, H., Ishikawa, T., and Suzuki, H. (2017). Clinical and Molecular Evidence of ABCC11 Protein Expression in Axillary Apocrine Glands of Patients with Axillary Osmidrosis. Int J Mol Sci 18(2), 417. doi: 10.3390/ijms18020417.

Wu, W. H., Ma, S., Lin, J. T., Tang, Y. W., Fang, R. H., and Yeh, F. L. (1994). Surgical treatment of axillary osmidrosis: an analysis of 343 cases. Plast Reconstr Surg 94(2), 288-294.

### FIGURE LEGENDS

FIG. 1. Febuxostat inhibited ABCC11 in vitro. (A) Expression of ABCC11 on membrane vesicles. Membrane vesicles were subjected to immunoblot analysis using an ABCC 11 antibody or anti-Na⁺/Kt⁺-ATPase antibody. Na⁺/K⁺-ATPase, a loading control. (B) Time-dependent increase in the dehydroepiandrosterone-sulfate (DHEAS) transport by ABCC11. The DHEAS transport into membrane vesicles was measured at the indicated periods with or without ATP; the ATP-dependent DHEAS transport was calculated by subtracting the DHEAS transport activity in the absence of ATP from that in the presence of ATP. Data are expressed as the mean±SD. n=3. Statistical analyses for significant differences between groups in each time point were performed using a two-sided t-test (††, P<0.01). (C) Dose-dependent inhibition of ABCC11-mediated DHEAS transport by febuxostat. The DHEAS transport activities were measured in the presence of febuxostat at the indicated concentrations for 5 min. Data are expressed as the mean±SD. n=4. Statistical analyses for significant differences were performed using Bartlett's test, followed by a Dunnett's test (**, P<0.01 vs 0 μM control).

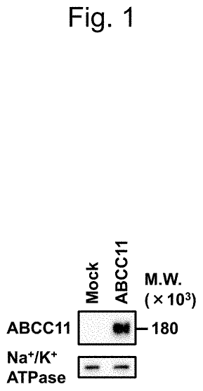

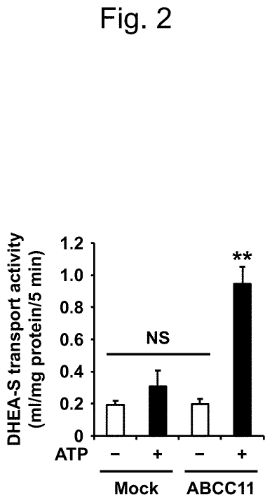

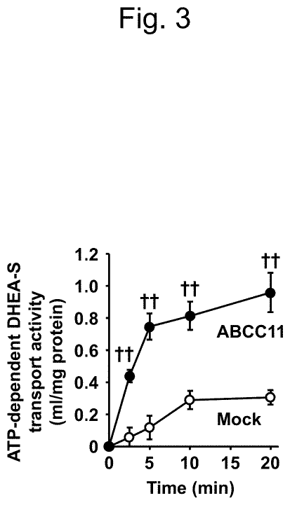

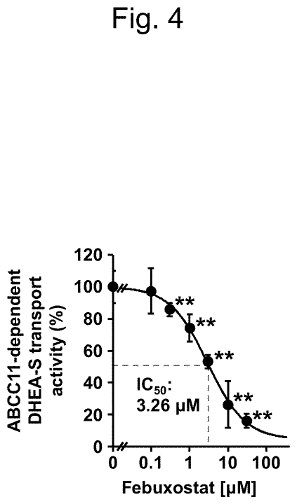

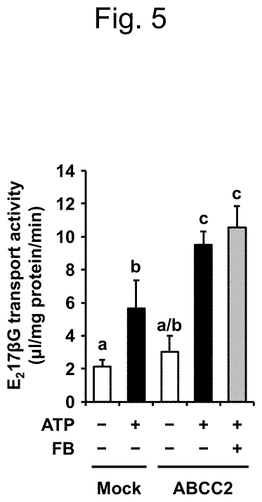

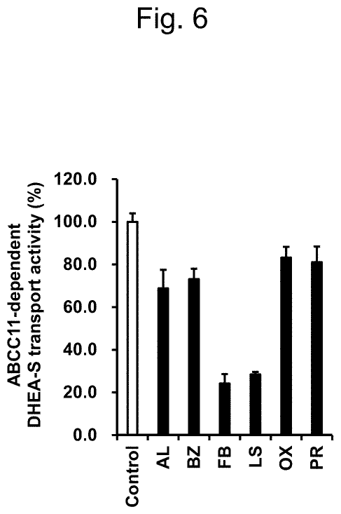

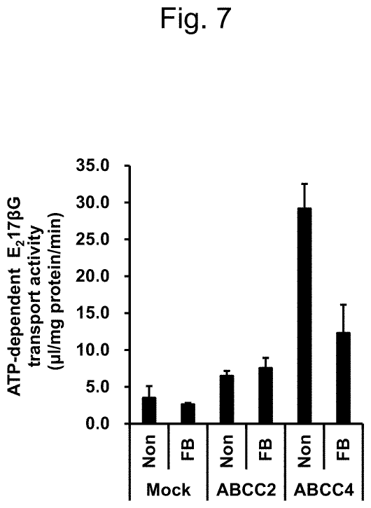

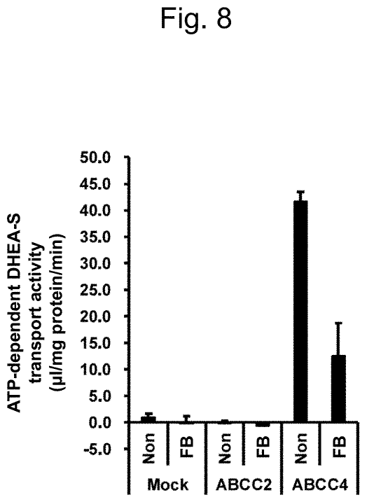

### SUPPLEMENTARY INFORMATION

### Supplementary Methods

### Materials

Critical materials and resources used in this study were summarized in Supplementary Table S1. Stock solution of febuxostat (Tokyo Chemical Industry, Tokyo, japan) was prepared with diethyl sulfoxide. Recombinant adenoviruses for the expression of the human ABCC11 WT (NCBI accession; NM_033151) or EGFP as a control were from our previous study (Toyoda et al., 2017). After the purification by a CsCl gradient ultracentrifugation method, the adenovirus titer was determined by using an. Adeno-X™ Rapid Titer Kit (Clontech Laboratories, Palo Alto, Calif., USA) according to manufacturer's instruction; the adenoviruses were stored at −80° C. until use. All the other chemicals used were commercially available and of analytical grade.

### Cell Culture

Human embryonic kidney 293 (HEK293)-derived 293A cells were maintained in Dulbecco's Modified Eagle's Medium (Nacalai Tesque, Kyoto, Japan) supplemented with 10% fetal bovine serum (Biowest, Nuaillé, France), and 1% penicillin-streptomycin (Nacalai Tesque), 2 mM L-Glutamine (Nacalai Tesque), and 1×Non-Essential Amino Acid (Life Technologies, Tokyo, Japan) at 37° C. in a humidified atmosphere of 5% (v/v) CO₂ in air. Adenovirus infection was performed as described previously (Toyoda et al., 2017).

### Preparation of ABCC11-Expressing Plasma Membrane Vesicles

Membrane vesicles were prepared from 293A cells infected with the ABCC11-expressing or EGFP-expressing (control) adenovirus as described previously (Toyoda et al., 2017). Obtained plasma membrane vesicles were rapidly frozen in liquid N₂ and kept at −80° C. until used. Protein concentration of the plasma membrane vesicles was quantified using a BCA Protein Assay Kit (Pierce, Rockford, Ill., USA) with bovine serum albumin (BSA) as a standard according to the manufacturer's protocol.

### Immunoblotting

Expression of ABCC11 protein in the plasma membrane vesicles was examined by immunoblotting as described previously (Toyoda et al., 2009; Toyoda et al,, 2017) with minor modifications. In brief, the prepared samples were electrophoretically separated on poly-acrylamide gels and then transferred to Hybond® ECL™ nitrocellulose membrane (GE Healthcare, Buckinghamshire, UK) by electroblotting at 15 V for 70 min. For blocking, the membrane was incubated in Tris-buffered saline containing 0.05% Tween 20 and 5% skim milk (TBST-skim milk), at 4° C. overnight. Blots were probed with the anti-ABCC11 antibody (M8I-74; Abeam, Cambridge, Mass., USA; diluted 200 fold) and a rabbit anti-Na⁺/K⁺-ATPase α antibody (sc-28800; Santa Cruz Biotechnology, Santa Cruz, Calif., USA; diluted 1,000 fold) followed by incubation with a goat anti-rat immunoglobulin G (IgG)-horseradish peroxidase (HRP)-conjugated antibody (NA935V; GE Healthcare; diluted 2,000 fold) and a donkey anti-rabbit IgG-HRP-conjugated antibody (NA934V; GE Healthcare; diluted 3,000 fold), respectively. All antibodies were used in TBST-skim milk. HRP dependent luminescence was developed using the ECL™ Prime Western Blotting Detection Reagent (GE Healthcare) and detected using a multi-imaging Analyzer Fusion Solo 4™ system (Vilber Lourmat, Eberhardzell, Germany).

### Vesicle Transport Assay

Experiments to study the in vitro transport of [1,2,6,7-³H(N)]-dehydroepiandrosterone sulfate (DHEAS) (Perkin-Elmer Japan, Tokyo, Japan), an ABCC11 substrate (Chen et al., 2005), into ABCC11-expressing and control vesicles were performed by using a rapid filtration technique (Toyoda et al., 2017; Toyoda et al., 2019) with a minor modification. In brief, the membrane vesicles were incubated with 100 nM of [1,2,6,7-³H(N)]-DHEAS in the reaction mixture (10 mM Tris/HCl, 250 mM sucrose, 10 mM MgCl₂, 10 mM creatine phosphate, 1 mg/mL creatine phosphokinase, pH 7.4, and 50 mM ATP or AMP as a substitute of ATP) for indicated periods at 37° C. with or without febuxostat at indicated concentrations. Then, the radioactivity derived from the incorporated DHEAS was measured. To reduce background signals derived from nonspecifically adsorbed radiolabeled DHEAS on filter membrane (MU-Millipore Membrane, HAWP02500; 0.45 μm pore size and 25 mm diameter; Millipore, Tokyo, Japan) for the trapping of plasma membrane vesicles, the filter membranes were incubated with 2 μM of cholesterol (Wako Pure Chemical Industries, Tokyo, Japan) in ice-cold Stop buffer (250 mM Sucrose, 0.1 M NaCl, 2 mM EDTA, and 10 mM Tris-HCl, pH 7.4) before use. In this transport experiment, the transport activity in each group was calculated as incorporated clearance [μL/mg protein/min=incorporated level of DHEAS (disintegrations per minute (DPM)/mg protein/min)/DHEAS level in the incubation mixture (DPM/μL)]. ATP-dependent DHEAS transport was calculated by subtracting the DHEAS transport activity in the absence of ATP from that in the presence of ATP.

### Calculation of the Half-Maximal Inhibitory Concentration (IC₅₀) Values

To address IC₅₀ values of febuxostat against the DHEAS transport by ABCC11, the DHEAS transport activities were measured in the presence of febuxostat at several concentrations. Then, the ABCC11-mediated transport activities were expressed as a. percentage of control (100%). Based on the calculated values, fitting curves were obtained according to the following formula using the least-squares methods with the Excel 2019 (Microsoft, Redmond, Wash., USA) as described previously (Miyata et al., 2016):

Predicted value [%]=100−(Eₘₐₓ×Cⁿ/EC₅₀ⁿ+Cⁿ)

where, Eₘₐₓ is the maximum effect, EC₅₀ is the half maximal effective concentration, C is the concentration of test compound, and n is the sigmoid-fit factor. Finally, based on the results, IC₅₀ was calculated.

### Statistical Analyses

All statistical analyses were performed by using Excel 2019 (Microsoft Corp., Redmond, Wash., USA) with Statcel4 add-in software (OMS publishing Inc., Saitama, Japan). Different statistical tests were used for different experiments as described in the figure legends. Briefly, when analyzing multiple groups, the similarity of variance between groups was compared using Bartlett's test. When passing the test for homogeneity of variance, a Dunnett's test was used. In the case of a single pair of quantitative data, after comparing the variances of a set of data by F-test, unpaired Student's t-test was performed. Statistical significance was defined in terms of P values less than 0.05 or 0.01.

Supplementary Table S1 Key resources.

| | | |
|---|---|---|
| REAGENT or RESOURCE | SOURCE | IDENTIFIER |

Antibodies

| | | |
|---|---|---|
| Rat monoclonal anti-MRP8 (ABCC11) antibody [M8I-74] | Abcam | Cat# ab91452; RRID: AB_2049125 |
| Rabbit polyclonal anti-Na⁺/K⁺-ATPase α antibody | Santa Cruz Biotechnology | Cat# sc-28800; RRID: AB_2290063 |
| Goat anti-rat IgG-horseradish peroxidase (HRP)-conjugate | GE Healthcare | Cat# NA935V; RRID: AB_772207 |
| Donkey anti-rabbit IgG-horseradish peroxidase (HRP)-conjugate | GE Healthcare | Cat# NA934V; RRID: AB_772206 |

Chemicals, Peptides, and Recombinant Proteins

| | | |
|---|---|---|
| Dehydroepiandrosterone sulfate, sodium salt, [1,2,6,7-³H(N)]-(1 mCi/mmol) | PerkinElmer | Cat# NET860 |
| Clear-sol II | Nacalai Tesque | Cat# 09136-83 |
| Dimethyl Sulfoxide | Nacalai Tesque | Cat# 13445-74; CAS: 67-68-5 |
| Cholesterol | Wako Pure Chemical Industries | Cat# 034-03002; CAS: 57-88-5 |
| Febuxostat | Tokyo Chemical Industry | Cat# F0847; CAS: 144060-53-7 |

Critical Commercial Assays

| | | |
|---|---|---|
| Pierce™ BCA Protein Assay Reagent A | Thermo Fisher Scientific | Cat# 23223 |
| Pierce™ BCA Protein Assay Reagent B | Thermo Fisher Scientific | Cat# 23224 |
| PureLink™ HiPure Plasmid Filter Midiprep Kit | Thermo Fisher Scientific | Cat# K210015 |

Virus strains

| | | |
|---|---|---|
| ABCC11-expressing adenovirus | Toyoda et al, 2017 | N/A |
| EGFP-expressing adenovirus | Toyoda et al, 2017 | N/A |

Recombinant DNA

| | | |
|---|---|---|
| The complete human ABCC11 cDNA | Toyoda et al, 2009 | NCBI Reference Sequence: NM_033151 |

Experimental Models: Cell Lines

| | | |
|---|---|---|
| 293A | Invitrogen | R70507 |

Software and Algorithms

| | | |
|---|---|---|
| Excel 2019 | Microsoft | https://www.microsoft.com/ja-jp/ |
| Statcel4 add-in software | OMS publishing | http ://www.oms-publ.co.jp/ |

### Supplementary References

Chen, Z. S., Guo, Y., Belinsky, M. G., Kotova, E., and Kruh, G. D. (2005). Transport of bile acids, sulfated steroids, estradiol 17-beta-D-glucuronide, and leukotriene C4 by human multidrug resistance protein 8 (ABCC11). Mol Pharmacol 67(2), 545-557. doi: 10.1124/mol104.007138.

Miyata, H., Takada, T., Toyoda, Y, Matsuo, H., Ichida, K., and Suzuki, H. (2016). Identification of Febuxostat as a New Strong ABCG2 Inhibitor: Potential Applications and Risks in Clinical Situations. Front Pharmacol 7, 518. doi: 10.3389/fphar.2016.00518.

Toyoda, Y., Sakurai, A., Mitani, Y., Nakashima, M., Yoshiura, K., Nakagawa, H., et al. (2009). Earwax, osmidrosis, and breast cancer: why does one SNP (538G>A) in the human ABC transporter ABCC11 gene determine earwax type? FASEB J 23(6), 2001-2013. doi: 10.1096/fj.09-129098.

Toyoda, Y., Takada, T., Gomi, T., Nakagawa, H., Ishikawa, T., and Suzuki, H. (2017). Clinical and Molecular Evidence of ABCC11 Protein Expression in Axillary Apocrine Glands of Patients with Axillary Osmidrosis. Int J Mol Sci 18(2), 417. doi.: 10.3390/ijms18020417.

Toyoda, Y., Takada, T., and Suzuki, H. (2019). Inhibitors of Human ABCG2: From Technical Background to Recent Updates With Clinical implications. Front Pharmacol 10, 208. doi: 10.3389/fphar.2019.00208.

1. An inhibitor of multidrug resistance-associated proteins with 12 transmembrane domains comprising, as an active ingredient, a compound for treatment of hyperuricemia or a salt thereof.

2. An ABCC11 inhibitor comprising, as an active ingredient, a compound for treatment of hyperuricemia or a salt thereof.

3. The inhibitor according to claim 1, wherein the compound is represented by Formula I: 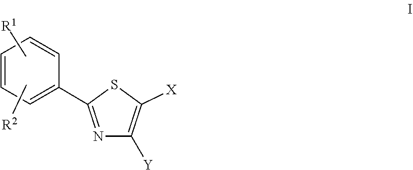
wherein
R¹ represents a halogen atom, a nitro group (—NO₂), a cyano group (—CN), a formyl group (—CHO), or a trifluoromethyl group (—CF₃),
R² represents a hydrogen atom or an alkoxy group having C₁₋₁₀ linear or branched alkyl group,
X represents a carboxyl group (—CO₂H), a carbamoyl group (—CONH₂), or an alkoxycarbonyl group having C₁₋₅ linear or branched alkoxy group, and
Y represents a hydrogen atom or a C₁₋₄ linear or branched alkyl group.

4. The inhibitor according to claim 3, wherein X represents a carboxyl group and Y represents a methyl group.

5. The inhibitor according to claim 3, wherein R² represents an alkoxy group having C₁₋₄ linear or branched alkyl group.

6. The inhibitor according to claim 3, wherein R¹ represents a cyano group.

7. The inhibitor according to claim 1, wherein the compound is represented by Formula II: 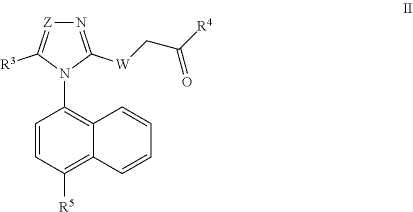
wherein
R³ represents a hydrogen atom, a halogen atom, a cyano group (—CN), a hydroxyl group (—OH), a nitro group (—NO₂), or an amino group (—NH₂),
R⁴ represents a hydroxyl group (—OH), an amino group (—NH₂), an alkoxy group having a C₁₋₆ linear or branched alkyl group, or a monoalkylamino or dialkylamino group substituted with a C₁₋₆ linear or branched alkyl group,
R⁵ represents a cyano group (—CN), a C₁₋₆ linear or branched alkyl group, or a C₃₋₅ cycloalkyl group,
Z represents a carbon or nitrogen atom, and
W represents a sulfur or oxygen atom.

8. The inhibitor according to claim 7, wherein R³ represents F, Cl, or Br.

9. The inhibitor according to claim 7, wherein R⁴ represents a hydroxyl group or an alkoxy group having C₁₋₄ linear or branched alkyl group.

10. The inhibitor according to claim 7, wherein R⁵ represents a cyano or cyclopropyl group.

11. The ABCC11 inhibitor according to claim 7, wherein Z represents a nitrogen atom and W represents a sulfur atom.

12. An inhibitor of multidrug resistance-associated proteins with 12 transmembrane domains comprising, as an active ingredient, a compound selected from the group consisting of Febuxostat, Lesinurad, and a salt thereof.

13. The inhibitor according to claim 1, which lowers a level of in vitro transport of small molecule substances mediated by multidrug resistance-associated proteins with 12 transmembrane domains in plasma membrane vesicles expressing multidrug resistance-associated proteins with 12 transmembrane domains to 80% or lower.

14. A pharmaceutical or cosmetic composition comprising the inhibitor according to claim 1.

15. A method for the prevention or treatment of axillary osmidrosis, comprising administering a composition of claim 14 to a patient in need thereof.

16. The method according to claim 15, wherein the composition is a topical preparation.

17. The method according to claim 16, wherein the topical preparation is in the form of an ointment, cream, emulsion, lotion, spray, powder, or gel.

18. The inhibitor according to claim 2, wherein the compound is represented by Formula I: 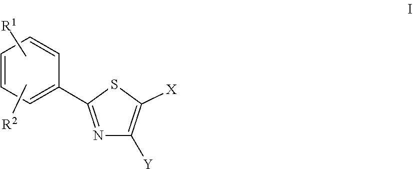
wherein
R¹ represents a halogen atom, a nitro group (—NO₂), a cyano group (—CN), a formyl group (—CHO), or a trifluoromethyl group (—CF₃),
R² represents a hydrogen atom or an alkoxy group having C₁₋₁₀ linear or branched alkyl group,
X represents a carboxyl group (—CO₂H), a carbamoyl group (—CONH₂), or an alkoxycarbonyl group having C₁₋₅ linear or branched alkoxy group, and
Y represents a hydrogen atom or a C₁₋₄ linear or branched alkyl group.

19. The inhibitor according to claim 2, wherein the compound is represented by Formula II: 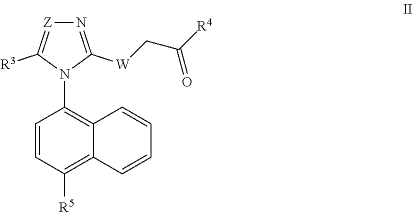
wherein
R³ represents a hydrogen atom, a halogen atom, a cyano group (—CN), a hydroxyl group (—OH), a nitro group (—NO₂), or an amino group (—NH₂),
R⁴ represents a hydroxyl group (—OH), an amino group (—NH₂), an alkoxy group having a C₁₋₆ linear or branched alkyl group, or a monoalkylamino or dialkylamino group substituted with a C₁₋₆ linear or branched alkyl group,
R⁵ represents a cyano group (—CN), a C₁₋₆ linear or branched alkyl group, or a C₃₋₅ cycloalkyl group,
Z represents a carbon or nitrogen atom, and
W represents a sulfur or oxygen atom.

19. The inhibitor according to claim 19, wherein X represents a carboxyl group and Y represents a methyl group.
